(19) United States
(12) Patent Application Publication
(10) Pub. No.: US 2016/0151256 A1
(43) Pub. Date: Jun. 2, 2016

(54) ORAL COMPOSITION

(71) Applicant: Janice Rae Goodman, Toronto (CA)

(72) Inventor: Janice Rae Goodman, Toronto (CA)

(21) Appl. No.: 14/951,829

(22) Filed: Nov. 25, 2015

Related U.S. Application Data
(60) Provisional application No. 62/085,384, filed on Nov. 28, 2014.

(51) Int. Cl. A61K 8/35; A61K 8/92; A61K 8/31; A61Q 11/00; A61K 8/34

(52) U.S. Cl. CPC A61K 8/35; A61Q 11/00; A61K 8/345; A61K 8/31; A61K 8/922; A61K 2800/30

(57) ABSTRACT

According to one aspect, an oral composition comprises a matrix comprising glycerin, an effective amount of limonene in the matrix, an effective amount of carvone in the matrix, and an effective amount of pinene in the matrix.

### CROSS REFERENCE TO RELATED APPLICATIONS

This application claims the benefit of U.S. Provisional Application No. 62/085,384, entitled ORAL COMPOSITION, filed on Nov. 28, 2014, which is incorporated herein by reference in its entirety.

### FIELD

The disclosure relates to compositions which may be applied to the oral cavity. More specifically, the disclosure relates to gels for application to the surfaces of the oral cavity, and which provide cosmetic and/or health benefits to the oral cavity.

### BACKGROUND

Good oral hygiene can keep the mouth and teeth clean, and can help to prevent oral pathologies such as halitosis, tooth staining, plaque formation, biofilm formation, gingivitis, periodontal disease, and dental caries. These pathologies may result in tooth decay and lead to tooth loss if not treated or prevented. Further, other complications may result from poor oral hygiene including an increased risk of systemic diseases such as cardiovascular disease, diabetes, and bacterial pneumonia.

Regular teeth cleaning through tooth brushing, using a gel or a paste, and flossing in order to remove dental plaque and tartar is one of the most common ways through which people maintain oral hygiene to prevent dental pathologies. Plaque is a bacterial film or biofilm that forms on the surface of teeth and gums that can give off acids and toxins, which have deleterious effects on oral tissues and can lead to dental pathologies such as caries and gingivitis.

The use of oral compositions such as mouth wash and cleaning the tongue regularly are also ways in which one can maintain oral health by reducing the levels of oral bacterial species. However, as oral pathologies remain relatively common despite the use of known oral hygiene compositions and methods.

### SUMMARY

The following summary is intended to introduce the reader to various aspects of the applicant's teaching, but not to define any invention.

The present application relates to oral compositions, such as oral gels, which may be applied to the surfaces of the oral cavity, and which may adhere to, form a film on, and/or coat these surfaces. The oral gels may in some examples include a base or matrix, and D-limonene in the base or matrix. The base or matrix may include or consist essentially of glycerin. The oral gels may further include carvone and/or pinene in the matrix. The D-limonene, carvone, and/or pinene may be provided by one or more essential oils, including but not limited to essential oils of lime, spearmint, mandarin, orange, sweet orange, lemon, peppermint, grapefruit, cornmint, cinnamon, coriander, caraway, or combinations thereof.

The oral gels may provide one or more cosmetic or health benefits. It is believed that such benefits may include, but are not limited to, the prevention, reduction, and/or inhibition of biofilm formation, staining, plaque formation, gingivitis, periodontal disease, caries, halitosis, dry mouth, and combinations thereof; increased shininess of the teeth; and soothing of painful lesions in the oral cavity (e.g. cold sores).

According to one aspect, an oral composition comprises a matrix comprising glycerin, an effective amount of limonene in the matrix to inhibit biofilm formation and/or inhibit staining, an effective amount of carvone in the matrix to provide flavor and/or inhibit biofilm formation, and an effective amount of pinene in the matrix to provide flavor and/or inhibit biofilm formation.

According to another aspect, an oral composition comprises a matrix comprising glycerin, at least 150 mg lime essential oil per 100 mL of matrix, and at least 30 mg spearmint essential oil per 100 mL of matrix.

According to another aspect, an oral composition comprises a matrix of glycerin, and at least one essential oil or constituent of essential oil in the matrix. The at least one essential oil or constituent of essential oil comprises limonene, carvone, and pinene.

According to another aspect, an oral composition comprises a matrix comprising glycerin, and an effective amount of limonene in the matrix to inhibit biofilm formation and/or inhibit staining. The oral composition may not include an effective amount of whitening agent.

The matrix may consist essentially of glycerin.

The oral compositions may in some examples comprise at least about 0.3% D-limonene (w/v), or between 0.3% and 5% D-Limonene.

The oral compositions may be a gel or a liquid.

The oral compositions may comprise, but are not limited to, at least one essential oil selected from the group consisting of lime, lemon, orange, sweet orange, mandarin, grapefruit, spearmint, cornmint, peppermint, coriander, caraway, and cinnamon.

According to another aspect, a method for oral care comprises applying any of the above oral compositions to at least one surface of the oral cavity. The method can include applying an oral composition comprising a matrix of glycerin and at least one essential oil in the matrix to at least one surface of the oral cavity. The at least one essential oil can include limonene, carvone, and pinene.

The at least one surface of the oral cavity may be selected from the group consisting of the teeth, a tongue, a roof of a mouth, gum tissue, and under the tongue.

### BRIEF DESCRIPTION OF THE DRAWINGS

The detailed description will make reference to the drawings in which:

FIG. 1 provides a graphical representation of the data obtained during an in vivo study. In the study, dental patients were asked to use the oral composition described in Example 2, below, and to report whether the composition had an acceptable taste, resulted in cleaner teeth, resulted in smoother and shinier teeth, and whether they experienced fresher breath upon using the composition. The data shown is a ranking from best (0) to worst (10).

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any invention disclosed in an apparatus or process described below and for which an exclusive right is not granted by issuance of this patent application may be the subject matter of another protective instrument, for example, a continuing patent application, and the applicants, inventors or owners do not intend to abandon, disclaim or dedicate to the public any such invention by its disclosure in this document.

The present application relates to compositions or formulations that can be delivered or applied to the oral cavity, and that may provide cosmetic and/or health benefits to the oral cavity.

In some examples, the compositions may be in the form of a liquid. In some examples, the compositions may be in the form of a gel, such as a generally viscous gel that may adhere to, coat, or form a film on the surfaces of the oral cavity (such as the teeth, the tongue, the gums, the roof of the mouth (for example, the hard and soft palate), and under the tongue). As used herein, the term “gel” includes generally viscous liquids (i.e. sufficiently viscous to adhere to, coat, or form a film on the surfaces of the oral cavity) that are transparent or translucent. In other examples, the composition may be in the form of a paste. As used herein, the term “paste” refers to a substance that has viscosity and flow characteristics comparable to those of a non-Newtonian liquid.

In some examples, the compositions may be referred to as a tooth gloss or a dental gloss. The tooth gloss may be painted, brushed, rubbed, or otherwise applied onto the teeth. The composition may be applied to the teeth with a brush or other applicator, may be applied to the teeth with the fingers, or may be applied to the tongue and then subsequently applied to the teeth with the tongue.

In other examples, the composition may be applied elsewhere in the mouth, for example to the tongue, under the tongue, to the roof of the mouth including the hard or soft palate, or to gums. The composition may in some examples be allowed to passively disperse through the oral cavity, or may be actively dispersed with the tongue and saliva.

In some examples, the composition may be provided in a container such as a vial, together with an applicator for applying the composition to the oral cavity. It is also contemplated herein that the composition and applicator may be provided in the form of a commercially available kit.

In some examples, the composition may be relatively long lasting in the mouth. For example, the composition may last longer on the surfaces of the mouth than a typical oral rinse.

The composition includes a base or matrix. The base or matrix may include or consist essentially of glycerin. The glycerin may be vegetable based glycerin. The composition further includes an effective amount of D-Limonene in the matrix. D-limonene can reduce dental staining and biofilm formation.

It is believed that such a composition may provide one or more cosmetic and/or health benefits to the oral cavity. For example, it is believed that the glycerin may make the teeth appear shinier, and may protect the teeth from stains, plaque formation, biofilm formation, and caries. Furthermore, the glycerin provides a matrix that may be long-lasting in the oral cavity (e.g. as compared to an oral rinse), and that coats, forms a film on, and/or adheres to the various surfaces of the oral cavity, to allow for the delivery of D-Limonene to the various surfaces of the oral cavity. It is believed that the D-Limonene may serve to reduce, inhibit, or prevent staining, biofilm formation, and/or halitosis. It is further believed that these effects may be enhanced by the combination of D-limonene with glycerin, as the glycerin brings the D-limonene in close contact with the surfaces of the mouth for a relatively long period of time. It is further believed that the composition may serve to reduce, prevent, or inhibit gingivitis and periodontal disease. It is further believed that the composition may serve to soothe pain associated with lesions in the mouth, such as cold sores.

As used herein, the term “effective amount” refers to a sufficient quantity of a component contained in the composition to achieve its intended purpose.

Accordingly, in some examples, the composition may include at least 0.3% D-limonene (w/v), and more specifically between about 0.3% and 5% D-Limonene. Such amounts may be effective to reduce dental staining and biofilm formation.

In some examples, the composition may further include carvone. The carvone may serve to enhance flavor. It is believed that the carvone may also provide additional health benefits, such as antiseptic/antibacterial effects, as well as other health benefits. The glycerin allows for the delivery of the carvone to the surfaces of the oral cavity.

In some examples, the composition may include at least 0.3% carvone (w/v), and more specifically between about 0.3% and 5% carvone.

In some examples, the composition may further include pinene, such as alpha-pinene and/or beta-pinene. The pinene may serve to enhance flavor. It is believed that the pinene may also provide additional health benefits. The glycerin allows for the delivery of the pinene to the oral cavity.

In some examples, the composition may include at least 0.3% pinene (w/v), and more specifically between about 0.3% and 5% pinene.

In some examples, any of the D-Limonene, carvone, and pinene may be provided by an essential oil, or a combination of essential oils (i.e. the D-Limonene, carvone, and/or pinene can be constituents of one or more essential oils). For example, essential oil of lime may provide D-Limonene, alpha-pinene, and beta-pinene; essential oil of spearmint may provide carvone, D-Limonene, and small amounts of alpha-pinene and beta-pinene; essential oil of cornmint may provide D-Limonene, alpha-pinene, and beta-pinene; essential oil of lemon may provide D-limonene, and small amounts of alpha pinene. Various other essential oils that may provide any of the D-Limonene, carvone, and pinene include orange, sweet orange, mandarin, grapefruit, spearmint, cornmint, peppermint, caraway, coriander, and cinnamon.

In some particular examples, the composition may include or consist essentially of glycerin, and essential oils. In some particular examples the composition may include or consist essentially of glycerin, essential oil of lime (also referred to as lime oil), which may optionally be distilled lime oil, and essential oil of spearmint (also referred to as spearmint oil).

In some examples, the composition may include between about 5 and about 15 drops of essential oil per 100 mg of glycerin, more specifically between about 8 drops and about 10 drops of essential oil per 100 mg of glycerin (where 1 drop=approximately 30 mg or 50 μl).

In one particular example, the composition may include at least about 150 mg distilled lime oil and about 30 mg spearmint oil, per 100 mL of base. Such formulations may provide effective amounts of D-limonene, carvone, and/or pinene.

In some examples, the composition may further include a thickener. Examples of thickeners include, but are not limited to, gum agar and/or carbopol.

In some examples, the composition may include other additives, such as opaquers, or sparklers. One example of an opaquer includes titanium dioxide.

In some examples, the composition may include a whitening agent, such as a bleach. As used herein, the term “bleach” may include a number of known chemicals which remove color and which whiten or disinfect and are safe for oral use. Exemplary bleaching chemicals include, without limitation, peroxides such as hydrogen peroxide, carbamide peroxide or other compounds such as titanium dioxide. However, the composition preferably does not include any whitening agents. Such examples may be useful because they may be applied with increased frequency as compared to compositions that include whitening agents.

In some examples, the composition may include cinnemaldehyde, which may be provided by essential oil of cinnamon.

In alternative examples, the composition may include an alternative base or matrix, other than glycerin. Suitable bases or matrices are those that are safe for use in the mouth, are relatively long-lasting in the oral cavity (e.g. longer lasting than an oral rinse), that coat, adhere to, and/or form a film on the various surfaces of the oral cavity, and that serve to deliver at least D-limonene, and optionally the carvone and/or pinene, to the surfaces of the oral cavity.

The compositions disclosed herein may also include acceptable carriers, vehicles or diluents to permit oral administration. Suitable carriers, vehicles and diluents are described, for example, in Remington's Pharmaceutical Sciences (2003—20th edition) and in The United States Pharmacopeia: The National Formulary (USP 24 NF19) published in 1999. Suitable acceptable carriers, vehicles or diluents include essentially chemically inert and non-toxic compositions that do not interfere with the effectiveness of the composition. Examples of suitable pharmaceutical carriers include, but are not limited to, water, saline solutions, glycerol solutions, ethanol, N-(1(2,3-d ioleyloxy)propyl)N,N,N-trimethylammonium chloride (DOTMA), diolesylphosphotidyl-ethanolamine (DOPE), and liposomes. Such compositions should contain an effective amount of the components of the composition together with a suitable amount of carrier so as to provide for direct administration to a patient.

In use, the composition may be applied to the teeth regularly, intermittently, or sporadically. For example, the composition may be applied every few hours, or every few days, only once in a while, or as needed. The amount of the composition to be applied may vary and depends on a user's preference. In one embodiment, one or two drops of the composition may be applied at each dosing.

It is believed that the composition may also provide other health benefits aside from oral health benefits. For example, the product may ultimately be ingested, and it is believed that the ingested D-limonene, and optionally the carvone and/or pinene, may provide health benefits. Such benefits include, but are not limited to, antispasmotic effects, antiseptic effects, antifungal effects, antibacterial effects, antiviral effects, and anticancer effects.

### EXAMPLES

### Example 1

A composition was prepared with the following make up:
100 ml Glycerin
150 mg Essential oil of lime (distilled)
30 mg Essential oil of spearmint
45 mg Essential oil of mandarin
45 mg Essential oil of sweet orange
30 mg Essential oil of lemon

The composition had the consistency of a sticky gel, or a viscous liquid.

The composition was tested by dental patients. The patients were instructed to apply one or more drops to the surfaces of the oral cavity as needed.

One or more of the patients made the following observations regarding the composition:
The composition was relatively long lasting in the mouth, and coated the teeth and other surfaces of the mouth.
The composition improved the taste of the mouth.
The composition improved the feeling of having a dry mouth, and as a result the patients did not constantly feel the need to drink water.
The composition improved the breath.

The composition made the teeth feel cleaner.
The composition coated the tongue and thereby soothed a cold sore on the tongue.
The composition made the patient feel less anxious in the dental chair.
The composition helped to settle a “nervous stomach”.

It is believed that with longer term use, the composition may also reduce, prevent, or inhibit one or more of staining, plaque formation, biofilm formation, gingivitis, periodontal disease, and caries.

### Example 2

A composition was prepared with the following make up:
100 ml Glycerin
150 mg Essential oil of lime (distilled)
30 mg Essential oil of spearmint
30-45 mg Essential oil of mandarin
60 mg Essential oil of sweet orange
30-45 mg Essential oil of lemon

The composition was tested by thirty dental patients. The patients were instructed to apply one or more drops to the surfaces of the oral cavity using a finger, toothbrush or other applicator, or directly into the mouth without an applicator, as needed, and to dispense the composition around the mouth using saliva. The patients were instructed to repeat this protocol once or twice a day.

Table 1 lists the results reported from patients who participated in this study aimed at assessing the effects of using the oral composition described above. Patients were asked to report whether, after use, they found the taste to be acceptable, whether they experienced cleaner teeth, whether they experienced smoother and shinier teeth and, finally, whether they experienced fresher breath. The data is reported as a percentage and absolute number of patients who either strongly agreed, agreed, were neutral, disagreed, or strongly disagreed with the statement found in the left column.

TABLE 1
Patient response following use of the oral composition

| | | | | | | Total |
|---|---|---|---|---|---|---|
| | | | | | | Number |
| | | | | | | of |
| | Strongly | | | | Strongly | Respon- |
| | Agree | Agree | Neutral | Disagree | Disagree | dents |
| Acceptable | 37.93% | 31.03% | 24.14% | 6.90% | 0.00% | 29 |
| taste | 11 | 9 | 7 | 2 | 0 | |
| Cleaner | 33.33% | 43.33% | 13.33% | 10.00% | 0.00% | 30 |
| teeth | 10 | 13 | 4 | 3 | 0 | |
| Smoother | 33.33% | 43.33% | 13.33% | 10.00% | 0.00% | 30 |
| and shinier | 10 | 13 | 4 | 3 | 0 | |
| teeth | | | | | | |
| Fresher | 27.59% | 44.83% | 24.14% | 3.45% | 0.00% | 29 |
| breath | 8 | 13 | 7 | 1 | 0 | |

As depicted by the in vivo data provided in Table 1, the majority of patients (20 of 29 respondents) reported that they strongly agree or agree that the taste is acceptable. The majority of patients (23 of 30 respondents) reported that they strongly agree or agree that they experienced cleaner teeth and smoother and shiner teeth following use of the composition. The majority of patients (21 of 29) also reported that they strongly agree or agree that they experienced fresher breath following use of the composition.

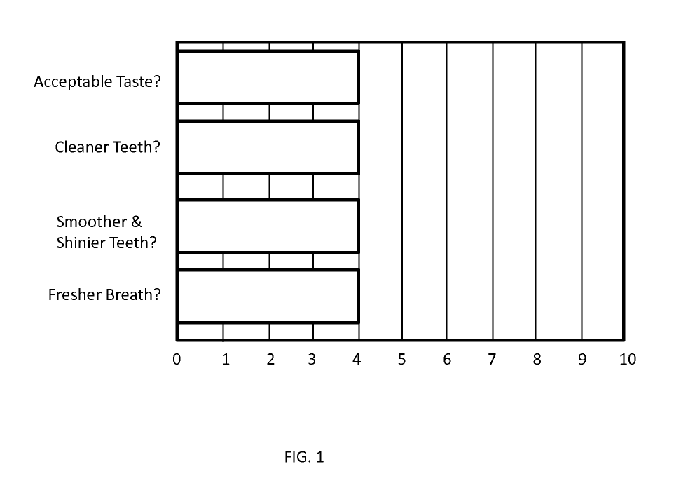

Further, FIG. 1 depicts graphically the results of the patient study. Patients were asked to use the oral composition and report whether the composition had an acceptable taste, resulted in cleaner teeth, resulted in smoother and shinier teeth, and whether they experienced fresher breath upon using the composition. The 30 patients involved in the study reported, on average, 4 out of 10, wherein the ranking is from best (0) to worst (10) for each of the parameters assessed (acceptable taste, cleaner teeth, smoother and shinier and fresher breath).

These data indicate that the composition and variants described herein may assist subjects in need thereof to experience fresher breath, cleaner teeth, and smoother and shinier teeth, while having an acceptable taste.

As clean teeth is a key factor in maintaining oral hygiene, it is believed that long term use of the oral compositions disclosed herein may assist in maintaining oral hygiene and in preventing halitosis, tooth staining, plaque formation, biofilm formation, gingivitis, periodontal disease, and caries. Further, the oral compositions disclosed may also assist patients cosmetically to obtain or maintain desirable tooth texture, namely smooth and shiny teeth. Smooth and shiny teeth may also provide a hygienic benefit to subjects by preventing or slowing the attachment of bacteria or biofilm formation.

While the above description provides examples of one or more processes or apparatuses, it will be appreciated that other processes or apparatuses may be within the scope of the accompanying claims.

1. An oral composition comprising:
a) a matrix comprising glycerin;
b) an effective amount of limonene in the matrix to inhibit biofilm formation and/or inhibit staining;
c) an effective amount of carvone in the matrix to provide flavor; and
d) an effective amount of pinene in the matrix to provide flavor.

2. The oral composition of claim 1, wherein the matrix consists essentially of glycerin.

3. The oral composition of claim 1, wherein the oral composition comprises at least 5% D-limonene (w/v).

4. The oral composition of claim 1, wherein the oral composition does not include an effective amount of whitening agents.

5. The oral composition of claim 1, wherein the oral composition is a gel.

6. An oral composition comprising:
a) a matrix comprising glycerin; and
b) at least 150 mg lime essential oil per 100 mL of matrix; and
c) at least 30 mg spearmint essential oil per 100 mL of matrix.

7. The oral composition of claim 6, wherein the matrix consists essentially of glycerin.

8. The oral composition of claim 6, wherein the oral composition further comprises at least 5% D-limonene (w/v).

9. The oral composition of claim 6, wherein the oral composition does not include an effective amount of whitening agents.

10. The oral composition of claim 6, wherein the oral composition is a gel.

11. An oral composition comprising:
a) a matrix of glycerin; and
b) at least one essential oil constituent in the matrix, the at least one essential oil constituent comprising limonene, carvone, and pinene, the at least one essential oil constituent present in an effective amount to inhibit staining and/or inhibit biofilm formation and/or inhibit halitosis.

12. The oral composition of claim 11, wherein the matrix consists essentially of glycerin.

13. The oral composition of claim 11, wherein the oral composition comprises at least 5% D-limonene (w/v).

14. The oral composition of claim 11, wherein the oral composition does not include an effective amount of whitening agents.

15. The oral composition of claim 11, wherein the oral composition is a gel.

16. The oral composition of claim 11, wherein the oral composition comprises at least one essential oil selected from the group consisting of lime, lemon, orange, sweet orange, mandarin, grapefruit, spearmint, cornmint, peppermint, and cinnamon.

17. An oral composition comprising:
a) a matrix comprising glycerin; and
b) an effective amount of limonene in the matrix to inhibit biofilm formation and/or inhibit staining;
wherein the oral composition does not include an effective amount of whitening agent.

18. The oral composition of claim 17, wherein the matrix consists essentially of glycerin.

19. The oral composition of claim 17, wherein the oral composition comprises at least 5% D-limonene (w/v).

20. The oral composition of claim 17, wherein the oral composition is a gel.

21. A method for oral care comprising:
applying an oral composition to at least one surface of the oral cavity, the oral composition comprising a matrix of glycerin and at least one essential oil constituent in the matrix, the at least one essential oil constituent comprising limonene, carvone, and pinene.

22. The method of claim 21, wherein the at least one surface of the oral cavity is selected from the group consisting of a tooth, a tongue, a roof of a mouth, gum tissue, and under the tongue.
